(19) United States
(12) Patent Application Publication
(10) Pub. No.: US 2022/0390409 A1
(43) Pub. Date: Dec. 8, 2022

(54) FLUID TRANSFER SYSTEM AND METHOD OF FORMING THE SAME

(71) Applicant: Agency for Science, Technology and Research, Singapore (SG)

(72) Inventors: Yu Chen, Singapore (SG); Weiguo Chen, Singapore (SG); Ming-Yuan Cheng, Singapore (SG); Ruiqi Lim, Singapore (SG)

(21) Appl. No.: 17/778,335

(22) PCT Filed: Nov. 10, 2020
(86) PCT No.: PCT/SG2020/050646

§ 371 Date: May 19, 2022

(30) Foreign Application Priority Data
Nov. 20, 2019 (SG) 10201910920S

(51) Int. Cl. G01N 27/333

(52) U.S. Cl. CPC G01N 27/333; G01N 2033/245

(57) ABSTRACT

Various embodiments may relate to a fluid transfer system. The fluid transfer system may include a chamber containing a fluid absorbing material in contact with a device. The fluid transfer system may also include a reservoir including rows of bendable support members, the reservoir connected to the chamber for transport of a fluid from the reservoir to the fluid absorbing material. The fluid transfer system may further include a mass including rows of protrusions configured to engage with the rows of bendable support members, the mass configured to be arranged in the reservoir such that forces acting on the mass include a gravitational force on the mass, a floating force exerted by the fluid on the mass, and a contact force exerted by the rows of bendable support members on the mass.

### CROSS-REFERENCE TO RELATED APPLICATION

This application claims the benefit of priority of Singapore application No. 10201910920S, filed Nov. 20, 2019, the contents of which being hereby incorporated by reference in its entirety for all purposes.

### TECHNICAL FIELD

Various embodiments of this disclosure may relate to a fluid transfer system. Various embodiments of this disclosure relate may relate to a method of forming a fluid transfer system.

### BACKGROUND

Sometimes, there is a need to keep the devices or materials moist for a long time. For example, electrodes for measuring pH may be required to be stored in buffer solution when not in use. Usually, this can be achieved if the devices or materials can be stored in an enclosure or do not need to be always exposed to air (there is minimum evaporation). However, in some situations, the device or materials need to be exposed to an ambient environment, and the evaporation of the water would require constant refilling of the water or moisture.

Traditionally, monitoring soil means going out to the field, taking soil samples, processing and doing measurements, followed by evaluating the measurements based on existing knowledge of the soil. The development of technology today makes it possible to remotely track soil parameters, such as -moisture, temperature, pH, salinity and so on, with much higher accuracy.

For soil nutrition monitoring in the farm, ion concentrations may need to be monitored for a long time or may need to be sampled many times. In such a case, a device which can function continuously without a sample preparation process will be beneficial. It may also be required to keep the sensor surface wet and enable the target ions to be able to be transferred and detected anytime.

### SUMMARY

Various embodiments may relate to a fluid transfer system. The fluid transfer system may include a chamber containing a fluid absorbing material in contact with a device. The fluid transfer system may also include a reservoir including rows of bendable support members, the reservoir connected to the chamber for the transport of fluid from the reservoir to the fluid absorbing material. The fluid transfer system may further include a mass including rows of protrusions configured to engage with the rows of bendable support members, the mass configured to be arranged in the reservoir such that forces acting on the mass include a gravitational force on the mass, a floating force exerted by the fluid on the mass, and a contact force exerted by the rows of bendable support members on the mass.

Various embodiments may relate to a method of forming a fluid transfer system. The method may include providing a chamber containing a fluid absorbing material in contact with a device. The method may also include connecting a reservoir to the chamber for the transport of fluid from the reservoir to the fluid absorbing material, the reservoir including rows of bendable support members. The method may further provide a mass including rows of protrusions configured to engage with the rows of bendable support members, the mass configured to be arranged in the reservoir such that forces acting on the mass include a gravitational force on the mass, a floating force exerted by the fluid on the mass, and a contact force exerted by the rows of bendable support members on the mass.

### BRIEF DESCRIPTION OF THE DRAWINGS

In the drawings, like reference characters generally refer to the same parts throughout the different views. The drawings are not necessarily drawn to scale, emphasis instead generally being placed upon illustrating the principles of various embodiments. In the following description, various embodiments of the invention are described with reference to the following drawings.

FIG. 1 is a schematic illustrating a fluid transfer system according to various embodiments.

FIG. 2 is a schematic illustrating a method of forming a fluid transfer system according to various embodiments.

FIG. 3A is a schematic illustrating an initial operation of a passive fluid transfer system according to various embodiments.

FIG. 3B shows is a schematic illustrating a subsequent operation of the passive fluid transfer system shown in FIG. 3A according to various embodiments, when the mass slips past the second topmost row of flexible pads of the reservoir onto the third topmost row of flexible pads as water in the chamber is pumped out and evaporated from the fluid or water absorbing material.

FIG. 3C shows is a schematic illustrating a subsequent operation of the passive fluid transfer system shown in FIG. 3B according to various embodiments, when the mass slips past the third topmost row of flexible pads of the reservoir onto the fourth topmost row of flexible pads as water in the chamber is pumped out and evaporated from the fluid or water absorbing material.

FIG. 3D shows is a schematic illustrating a subsequent operation of the passive fluid transfer system shown in FIG. 3C according to various embodiments, when the mass slips past the fourth topmost row of flexible pads of the reservoir onto the fifth topmost row of flexible pads as water in the chamber is pumped out and evaporated from the fluid or water absorbing material.

FIG. 3E shows is a schematic illustrating a subsequent operation of the passive fluid transfer system shown in FIG. 3D according to various embodiments, when the mass slips past the fifth topmost row of flexible pads of the reservoir onto the bottommost row of flexible pads as water in the chamber is pumped out and evaporated from the fluid or water absorbing material.

FIG. 4 shows (a) a cross-sectional schematic of an ion sensor chip according to various embodiments (the chip may have a fluidic channel and ions may be dissolved and be carried to the electrodes through an opening) for use in conjunction with the passive fluid transfer system according to various embodiments; (b) a cross-sectional schematic of the ion sensor chip including a hydrogel for locking water and maintaining the humidity of the sensor electrodes according to various embodiments; and (c) a cross-sectional schematic of a test chamber for water retention experiment according to various embodiments.

FIG. 5A shows a plot of resistance (in kilo-ohms or kΩ) as a function of frequency (in hertz or Hz) showing the conductivity measurements of the sensing electrodes with hydrogel sealing according to various embodiments.

FIG. 5B shows a plot of resistance (in kilo-ohms or kΩ) as a function of frequency (in hertz or Hz) showing the conductivity measurements of the sensing electrodes without hydrogel sealing according to various embodiments.

FIG. 6 shows (a) a cross-sectional schematic of the ion sensor chip including hydrogel according to various embodiments at start of experiment (T=0); (b) a cross-sectional schematic of the ion sensor chip shown in (a) according to various embodiments after 40 hours (T=40 h); (c) a cross-sectional schematic of the ion sensor chip shown in (a) according to various embodiments after 112 hours (T=112 h); (d) a cross-sectional schematic of the ion sensor chip without hydrogel according to various embodiments at start of experiment (T=0); (e) a cross-sectional schematic of the ion sensor chip shown in (d) according to various embodiments after 40 hours (T=40 h); and (f) a cross-sectional schematic of the ion sensor chip shown in (d) according to various embodiments after 112 hours (T=112 h).

FIG. 7 shows schematics of three fluidic channels according to various embodiments, each with a different initial water level.

FIG. 8 shows (left) a schematic illustrating a cross-sectional side view of the passive water transfer system according to various embodiments; and (right) a schematic illustrating a cross-sectional front view of the passive water transfer system according to various embodiments.

FIG. 9 is a schematic illustrating a fluid transfer system according to various embodiments.

FIG. 10 is a schematic illustrating a fluid transfer system according to various embodiments.

FIG. 11 is a schematic illustrating a fluid transfer system according to various embodiments.

### DESCRIPTION

The following detailed description refers to the accompanying drawings that show, by way of illustration, specific details and embodiments in which the invention may be practiced. These embodiments are described in sufficient detail to enable those skilled in the art to practice the invention. Other embodiments may be utilized and structural, logical, and electrical changes may be made without departing from the scope of the invention. The various embodiments are not necessarily mutually exclusive, as some embodiments can be combined with one or more other embodiments to form new embodiments.

Embodiments described in the context of one of the methods or fluid transfer systems are analogously valid for the other methods or fluid transfer systems. Similarly, embodiments described in the context of a method are analogously valid for a fluid transfer system, and vice versa.

Features that are described in the context of an embodiment may correspondingly be applicable to the same or similar features in the other embodiments. Features that are described in the context of an embodiment may correspondingly be applicable to the other embodiments, even if not explicitly described in these other embodiments. Furthermore, additions and/or combinations and/or alternatives as described for a feature in the context of an embodiment may correspondingly be applicable to the same or similar feature in the other embodiments.

The fluid transfer system as described herein may be operable in various orientations, and thus it should be understood that the terms “top”, “bottom”, etc., when used in the following description are used for convenience and to aid understanding of relative positions or directions, and not intended to limit the orientation of the fluid transfer system.

In the context of various embodiments, the articles “a”, “an” and “the” as used with regard to a feature or element include a reference to one or more of the features or elements.

In the context of various embodiments, the term “about” or “approximately” as applied to a numeric value encompasses the exact value and a reasonable variance.

As used herein, the term “and/or” includes any and all combinations of one or more of the associated listed items.

Various embodiments may relate to a fluid transfer system (alternatively referred to as a passive fluid transfer system or a water transfer/retention system), as well as a method of forming the same. The system may be used to keep a device, such as an electrode of a sensor, functioning for a long time without drying out.

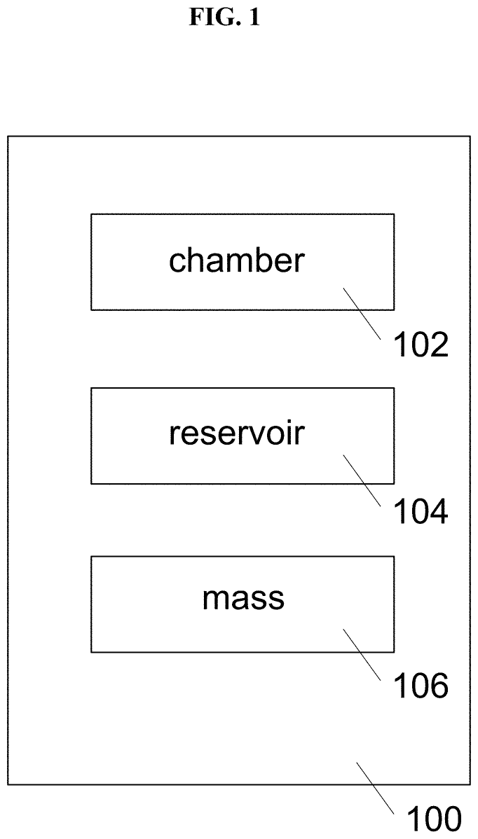

FIG. 1 is a schematic illustrating a fluid transfer system 100 according to various embodiments. The fluid transfer system 100 may include a chamber 102 containing a fluid absorbing material in contact with a device. The fluid transfer system 100 may also include a reservoir 104 including rows of bendable support members, the reservoir 104 connected to the chamber 102 for the transport of fluid from the reservoir to the fluid absorbing material. The fluid transfer system 100 may further include a mass 106 including rows of protrusions configured to engage with the rows of bendable support members, the mass 106 configured to be arranged in the reservoir 104 such that forces acting on the mass 106 include a gravitational force on the mass 106, a floating force exerted by the fluid on the mass 106, and a contact force exerted by the rows of bendable support members on the mass 106.

In other words, the fluid transfer system 100 may include a self-adjustable fluidic reservoir 104 containing a mass 106, and a chamber 102, e.g. a fluidic channel with a fluid absorbing material, e.g. water retention material, in fluidic communication with the reservoir 104 to keep the surrounding of the device, e.g. a sensing electrode wet.

For avoidance of doubt, FIG. 1 serves to illustrate some features of the system 100 according to various embodiments, and is not intended to limit the shape, sizes, arrangement, orientation etc. of these features.

The mass 106 may be a self-movable weight, meaning that there are no other forces acting on the mass 106 during operation of the system 100 except for the gravitational force, the floating force exerted by the fluid, and the contact force exerted by the rows of bendable support members on the mass 106.

The system 100/mass 106 may not be required to be manually controlled to provide fluid to the fluid absorbing material during operation. The gravitational force may be initially balanced by a sum of the floating force exerted by the fluid on the mass and the contact force exerted by the rows of bendable support members on the mass. In other words, the downwards gravitational force acting on the mass 106 may be initially balanced by a sum of the upwards floating force exerted by the fluid on the mass 106 and the upwards contact force exerted by the rows of bendable support members on the mass 106.

The gravitational force acting on the mass 106 may initially be balanced by a sum of the floating force exerted by the fluid on the mass 106 and the contact force exerted by the rows of bendable support members on the mass 106.

As the fluid is absorbed by the fluid absorbing material, a level of fluid in the reservoir 104 may decrease, and the floating force exerted on the mass 106 may decrease. In other words, the floating force exerted by the fluid on the mass 106 may decreases as the fluid travels from the reservoir 104 to the fluid absorbing material to replace the fluid lost via evaporation.

The rows of bendable support members may be configured such that a first row of bendable support members of the rows of bendable support members initially supporting a row of protrusions of the rows of protrusions bends as the floating force exerted by the fluid decreases, causing the gravitational force acting on the mass 106 to be more than the sum of the floating force exerted by the fluid on the mass 106 and the contact force exerted by the rows of bendable support members on the mass 106. In other words, as the fluid in the reservoir is transported to the chamber 102, a level of fluid in the reservoir 104 decreases (a gap between the level of fluid and the mass 106 increases), thereby reducing the floating force acting on the mass 106. The downwards gravitational force on the mass exceeds the sum of the upwards floating force and the upwards contact force, causing the rows of bendable support members to bend downwards.

Bending of the first row of bendable support members may allow the row of protrusions to move past the first row of bendable support members so that the row of protrusions is supported by the second row of bendable support members of the rows of bendable support members below the first row of bendable support members, thereby allowing the mass to move down relative to the reservoir 104. In other words, as the rows of bendable support members bends, the row of protrusions of the mass 106 initially supported by the first row of bendable support members “slips” past the first row of bendable support members onto the second row of bendable support members below the first row of bendable support members. In various embodiments, a further row of protrusions of the mass 106 which is initially supported by the second row of bendable support members may “slip” past the second row of bendable support members onto a third row of bendable support members below the second row of bendable support members. The other rows of protrusions of the mass 106 may also move past the corresponding rows of bendable support members. The bottom row of protrusions may move past the corresponding bottom row of bendable support members, and may no longer be supported by any bendable support members.

The floating force exerted by the fluid on the mass 106 may increase and the contact force exerted by the rows of bendable support members on the mass 106 may decrease as the mass moves down relative to the reservoir 104. The floating force may increase as the gap between the level of the fluid and the mass is restored (i.e. decreases), and the contact force may decrease as the number of bendable support members used to support the protrusions has decreased. There may be no external control to change the contact force exerted by the rows of bendable support members on the mass. In the current context, “bendable support members” may refer to members which are flexible or elastic. The bendable support members may be configured to bend or deform upon the application of an applied force (beyond a predetermined threshold). When the applied force is no longer provided, or if the applied force falls below the predetermined threshold, the bendable support member may return to its original shape. The bendable support members may be flexible pads. The bendable support members may include polydimethylsiloxane (PDMS), polyimide, parylene, and/or any other suitable materials.

The system 100 may be configured to automatically and slowly supply fluid to the fluid absorbing material by the repeated mechanism of the mass moving down relative to the reservoir 104 due to the lowering of the fluid level and the dynamic rebalancing of the gravitational force, the floating force and the contact force as the mass 106 moves down relative to the reservoir 104.

In various embodiments, the fluid may be water, or a pH buffer solution. In various embodiments, the fluid absorbing material may be a hydrophilic material e.g. a gel such as hydrogel or any other suitable water absorbent gel, cotton, or activated carbon. Examples of hydrogel may, for instance, be poly(acrylamide) (PAAm), poly(methacrylic acid) (PMAA), poly(N-isopropylacrylamide) (PNIPAM) etc.

The fluid absorbing material may draw fluid to the chamber 102 and may cause the lowering of the fluid level in the reservoir 104. The fluid absorbing material may thus exert a hydrogel withdrawing effect on the fluid. The fluid absorbing material may be continuously be supplied with the fluid from the reservoir 104, and may thus retain fluid in contact with the device. In various embodiments, the fluid absorbing material may surround the device.

In various embodiments, the device may be an ion sensor electrode (alternatively referred to as sensing electrode), or an ion sensor chip.

In various embodiments, the system 100 may include the device.

In various embodiments, the reservoir 104 may include one or more walls. The rows of bendable support members may extend from the one or more walls.

In various other embodiments, the reservoir 104 may include a lid and a column suspended from the lid. The rows of bendable support members may extend from the column.

In various embodiments, a number of rows of bendable support members may be equal to a number of rows of protrusions. In various other embodiments, a number of rows of bendable support members may not be equal to a number of rows of protrusions. For instance, the number of rows of bendable support members may be less than the number of rows of protrusions.

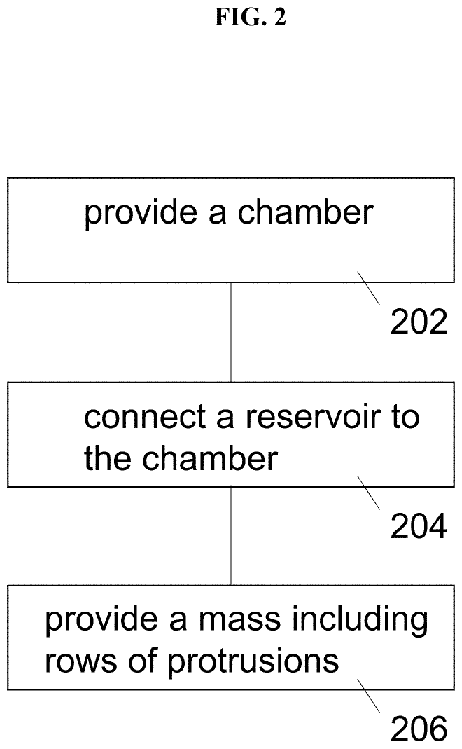

FIG. 2 is a schematic illustrating a method of forming a fluid transfer system according to various embodiments. The method may include, in 202, providing a chamber containing a fluid absorbing material in contact with a device. The method may also include, in 204, connecting a reservoir to the chamber for transport of a fluid from the reservoir to the fluid absorbing material, the reservoir including rows of bendable support members. The method may further include, in 206, providing a mass including rows of protrusions configured to engage with the rows of bendable support members, the mass configured to be arranged in the reservoir such that forces acting on the mass include a gravitational force on the mass, a floating force exerted by the fluid on the mass, and a contact force exerted by the rows of bendable support members on the mass.

In other words, the method may include connecting a reservoir rows of bendable support members and a chamber containing a fluid absorbing material. The method may also include providing a mass including rows of protrusions configured to engage with the rows of bendable support members.

For avoidance of doubt, FIG. 2 is not intended to limit the sequence of the various steps. For instance, in various embodiments, the mass may be provided after connecting the reservoir to the chamber, while in various other embodiments, the mass may be provided before connecting the reservoir to the chamber.

The gravitational force acting on the mass may initially be balanced by a sum of the floating force exerted by the fluid on the mass and the contact force exerted by the rows of bendable support members on the mass. The rows of bendable support members may be configured such that a first row of bendable support members of the rows of bendable support members initially supporting a row of protrusions of the rows of protrusions bends as the floating force exerted by the fluid decreases, causing the gravitational force acting on the mass to be more than the sum of the floating force exerted by the fluid on the mass and the contact force exerted by the rows of bendable support members on the mass. Bending of the first row of bendable support members may allow the row of protrusions to move past the first row of bendable support members so that the row of protrusions is supported by a second row of bendable support members of the rows of bendable support members below the first row of bendable support members, thereby allowing the mass to move down relative to the reservoir.

The floating force exerted by the fluid on the mass may decrease as the fluid travels from the reservoir to the fluid absorbing material to replace the fluid lost via evaporation.

The floating force exerted by the fluid on the mass may increase and the contact force exerted by the rows of bendable support members on the mass may decrease as the mass moves down relative to the reservoir.

In various embodiments, the fluid may be water, or a pH buffer solution. The fluid absorbing material may be a hydrophilic material, such as a hydrogel, cotton, or activated carbon.

In various embodiments, the method may include providing the device. The device may be an ion sensor electrode.

In various embodiments, the reservoir may include one or more walls. The rows of bendable support members may extend from the one or more walls.

In various other embodiments, the reservoir may include a lid, and a column suspended from the lid. The rows of bendable support members may extend from the column.

For on-site soil monitoring, the soil may be required to be in contact with sensing electrodes (i.e. ion sensor electrodes) or fluid absorbing materials (e.g. gel materials such as hydrogels) which cover the electrodes. The ions in the soil should be able to diffuse onto the electrodes and transfer to an electrical signal, which means the electrodes need to be wet and at the same time, exposed to the surrounding environment. Depends on the humidity and the temperature, the fluid or water inside this volume may evaporate over time. If no fluid or water is refilled into this volume, the ion sensor electrodes may be dried out and may not function properly.

A reservoir can be connected to the device/material through a chamber or fluidic channel to refill the water to the ion sensor electrode only when necessary. Conventionally, there are a few methods to enable the water refilling.

One approach is to attach a humidity/moisture sensor to the ion sensor electrode, and a valve to the fluidic channel. Whenever the humidity is lower than a threshold level, the valve can be triggered to open, and a certain amount of water can be delivered to the ion sensor electrode. This approach is similar to the closed loop controlled irrigation system. However, such an approach may be complicated, and the cost may be high.

The second approach is an open-loop controlled system, where a timer is used to open the valve for the water refilling. This method removes the sensor and makes the system cheap and less complicated.

For both of the above approaches, there is a need to have a battery to control the mechanic valve. This may increase the cost and also usually the water volume needed in the reservoir may be large. In contrast, various embodiments may not require an electrical energy source, such as a battery. Various embodiments may not require a reservoir with large volume due to the low consumption rate of water.

It is also possible to use capillary force to wick the water to the device to maintain the humidity. However, it is difficult to use a small amount of water. In addition, the clogging issue of the microfluidic channel may make the channel difficult to be used in a harsh environment for a long time.

Various embodiments may use fluid absorbing materials e.g. hydrophilic or water absorbent materials, such as hydrogel or other water absorbent gels, within a chamber surrounding the ion sensor electrode. Part of this chamber may be exposed to the environment where ions to be detected can diffuse onto the ion sensor electrode. By connecting this chamber to a water reservoir, whenever the water is evaporated, water from the reservoir may be consumed to ensure the moisture inside the chamber. Evaporation rate may depend on the temperature, exposed area size, and humidity of the environment. The fluid absorbing material may only withdraw enough water to keep the moisture. As a result, the consumption rate of water may be low.

When the water reservoir level is far below the hydrogel level, the withdrawal force of hydrogel may not be large enough to pump water to the device. In order to keep pumping water to the device, various embodiments may include a self-adjustable reservoir with a movable mass or weight which is supported by both floating force (due to the withdrawal force) and contact or supporting force from the bendable support members, such as flexible pads.

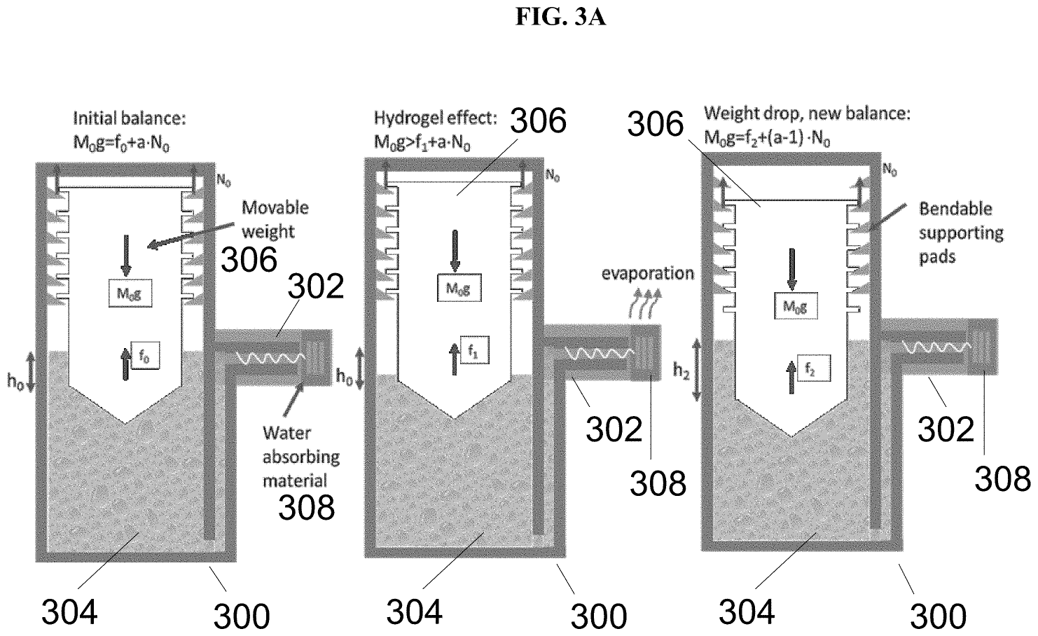

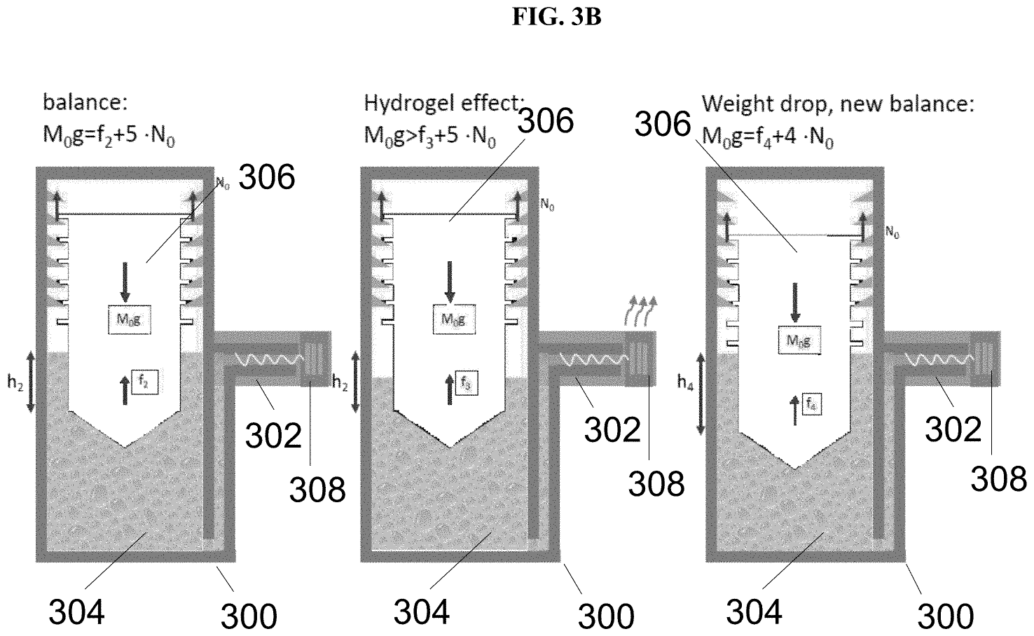

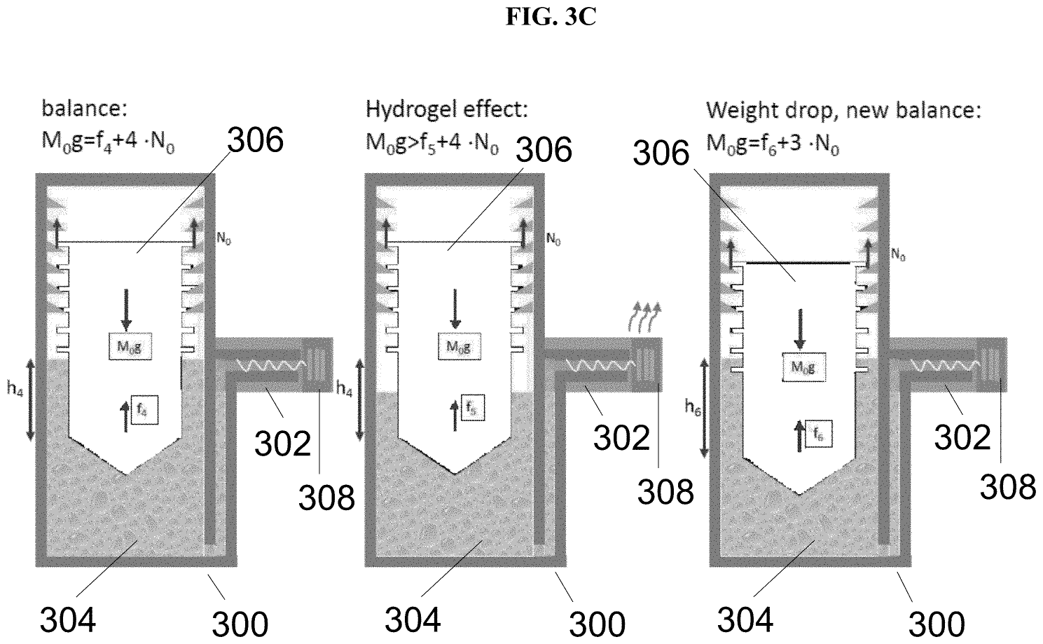

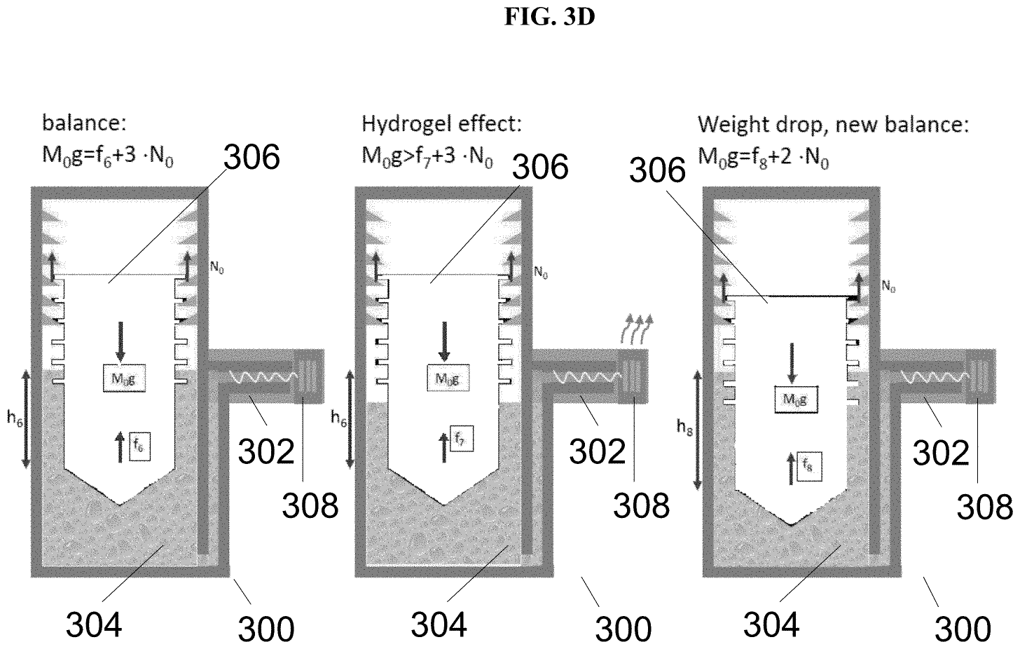

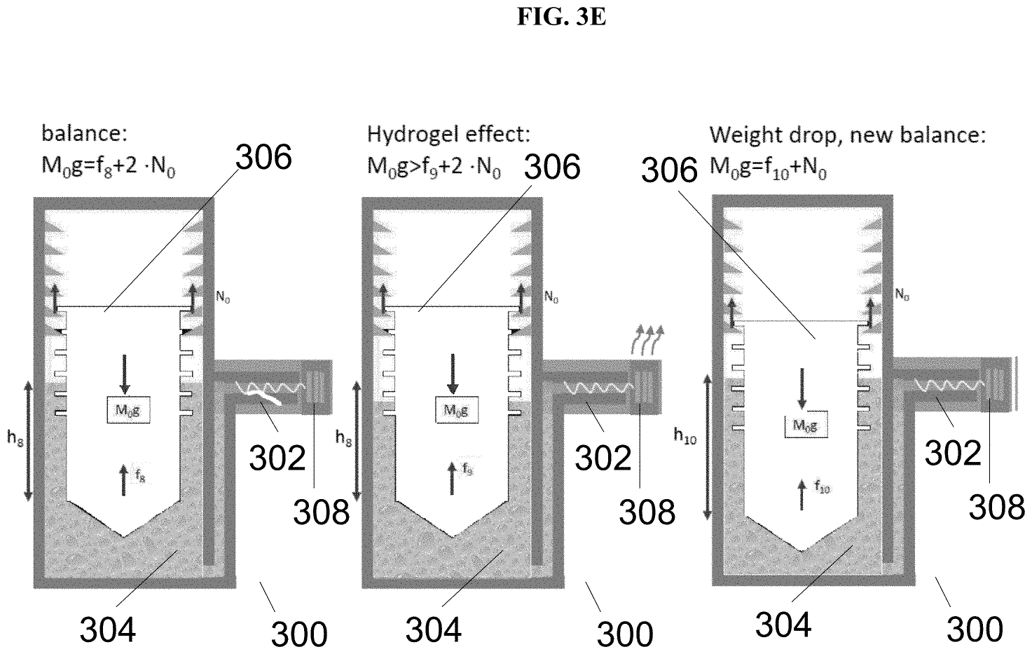

FIG. 3A is a schematic illustrating an initial operation of a passive fluid transfer system 300 according to various embodiments. At the initial state, the water level inside the reservoir is the same as the hydrogel. The movable weight or mass 306 (having weight M₀g, where M₀ is the mass, and g is the gravitational constant) is balanced by the floating force (f₀) exerted by the water and the contact force provided by 6 sets (as one example) of bendable support members, i.e. flexible or bendable supporting pads (6×N₀) of the reservoir 304. As shown in FIG. 3A, the flexible or bendable supporting pads may extend from the walls of the reservoir. The flexible or bendable supporting pads of the same row may face inwards, i.e. towards each other or one another. In contrast, the protrusions of the movable weight or mass 306 may face outwards. In various embodiments as illustrated in FIG. 3A, the number of rows of protrusions may be equal to the number of rows of bendable support members. In various other embodiments, the arrangement and the number of bendable support members and the arrangement and the number of the protrusions may be different from that depicted in FIG. 3A.

When the water in the chamber 302 evaporates through the exposed fluid or water absorbing material 308, e.g. hydrogel, the water from the reservoir 304 may be pumped out. As such, the water level inside the reservoir 304 may decrease, thereby decreasing the floating force (f₁) exerted by the water on the mass 306.

When the floating force decreases to a certain level such that the floating force exerted by the water and the contact forces exerted by the pads (6×N₀) cannot balance the weight of the mass 306, the mass 306 may fall pass the topmost row of the flexible or bendable supporting pads, thus pushing the water level inside the reservoir 304, which will increase the water level and the floating force. The floating force, f₂, may be larger than the initial force (f₀), but the supporting force from the pads is changed to only 5×N₀, so the combined floating force f₂ and the supporting force 5×N₀ may still balance with the weight (M₀g) of the mass 306. As shown in FIG. 3A, the bottommost row of protrusions of the mass 306 may no longer be supported by the pads.

The above process may be repeated as water in the chamber 302 is pumped out and evaporated from the fluid or water absorbing material 308 and as the mass 306 is falling down step by step.

FIG. 3B shows is a schematic illustrating a subsequent operation of the passive fluid transfer system 300 shown in FIG. 3A according to various embodiments, when the mass 306 slips past the second topmost row of flexible pads of the reservoir 304 onto the third topmost row of flexible pads as water in the chamber 302 is pumped out and evaporated from the fluid or water absorbing material 308. As shown in FIG. 3B, the weight (M₀g) of the mass 306 may initially be balanced by the supporting force 5×N₀ exerted by the flexible pads, as well as the contact force f₂. As water in the chamber 302 evaporates through the exposed fluid or water absorbing material 308, e.g. hydrogel, the water from the reservoir 304 may be pumped out. As such, the water level inside the reservoir 304 may decrease, thereby decreasing the floating force (f₃) exerted by the water on the mass 306. When the floating force decreases to a certain level such that the floating force exerted by the water and the contact forces exerted by the pads (5×N₀) cannot balance the weight of the mass 306, the mass 306 may fall pass the second topmost row of the flexible pads, thus increasing the floating force. The floating force, f₄, may be larger than the initial force (f₂), but the supporting force from the pads is changed to only 4×N₀, so the combined floating force f₄ and the supporting force 4×N₀ may still balance with the weight (M₀g) of the mass 306. As shown in FIG. 3B, the two bottommost rows of protrusions of the mass 306 may no longer be supported by the pads.

FIG. 3C shows is a schematic illustrating a subsequent operation of the passive fluid transfer system 300 shown in FIG. 3B according to various embodiments, when the mass 306 slips past the third topmost row of flexible pads of the reservoir 304 onto the fourth topmost row of flexible pads as water in the chamber 302 is pumped out and evaporated from the fluid or water absorbing material 308. As shown in FIG. 3C, the weight (M₀g) of the mass 306 may initially be balanced by the supporting force 4×N₀ exerted by the flexible pads, as well as the contact force f₄. As water in the chamber 302 evaporates through the exposed fluid or water absorbing material 308, e.g. hydrogel, the water from the reservoir 304 may be pumped out. As such, the water level inside the reservoir 304 may decrease, thereby decreasing the floating force (f₅) exerted by the water on the mass 306. When the floating force decreases to a certain level such that the floating force exerted by the water and the contact forces exerted by the pads (4×N₀) cannot balance the weight of the mass 306, the mass 306 may fall pass the third topmost row of the flexible pads, thus increasing the floating force. The floating force, f₆, may be larger than the initial force (f₄), but the supporting force from the pads is changed to only 3×N₀, so the combined floating force f₆ and the supporting force 3×N₀ may still balance with the weight (M₀g) of the mass 306. As shown in FIG. 3C, the three bottommost rows of protrusions of the mass 306 may no longer be supported by the pads.

FIG. 3D shows is a schematic illustrating a subsequent operation of the passive fluid transfer system 300 shown in FIG. 3C according to various embodiments, when the mass 306 slips past the fourth topmost row of flexible pads of the reservoir 304 onto the fifth topmost row of flexible pads as water in the chamber 302 is pumped out and evaporated from the fluid or water absorbing material 308. As shown in FIG. 3D, the weight (M₀g) of the mass 306 may initially be balanced by the supporting force 3×N₀ exerted by the flexible pads, as well as the contact force f₆. As water in the chamber 302 evaporates through the exposed fluid or water absorbing material 308, e.g. hydrogel, the water from the reservoir 304 may be pumped out. As such, the water level inside the reservoir 304 may decrease, thereby decreasing the floating force (f₇) exerted by the water on the mass 306. When the floating force decreases to a certain level such that the floating force exerted by the water and the contact forces exerted by the pads (3×N₀) cannot balance the weight of the mass 306, the mass 306 may fall pass the fourth topmost row of the flexible pads, thus increasing the floating force. The floating force, f₈, may be larger than the initial force (f₆), but the supporting force from the pads is changed to only 2×N₀, so the combined floating force f₈ and the supporting force 2×N₀ may still balance with the weight (M₀g) of the mass 306. As shown in FIG. 3D, the four bottommost rows of protrusions of the mass 306 may no longer be supported by the pads.

FIG. 3E shows is a schematic illustrating a subsequent operation of the passive fluid transfer system 300 shown in FIG. 3D according to various embodiments, when the mass 306 slips past the fifth topmost row of flexible pads of the reservoir 304 onto the bottommost row of flexible pads as water in the chamber 302 is pumped out and evaporated from the fluid or water absorbing material 308. As shown in FIG. 3E, the weight (M₀g) of the mass 306 may initially be balanced by the supporting force 2×N₀ exerted by the flexible pads, as well as the contact force f₈. As water in the chamber 302 evaporates through the exposed fluid or water absorbing material 308, e.g. hydrogel, the water from the reservoir 304 may be pumped out. As such, the water level inside the reservoir 304 may decrease, thereby decreasing the floating force (f₉) exerted by the water on the mass 306. When the floating force decreases to a certain level such that the floating force exerted by the water and the contact forces exerted by the pads (2×N₀) cannot balance the weight of the mass 306, the mass 306 may fall pass the fifth topmost row of the flexible pads, thus increasing the floating force. The floating force, f₁₀, may be larger than the initial force (f₈), but the supporting force from the pads is changed to only N₀, so the combined floating force f₁₀ and the supporting force N₀ may still balance with the weight (M₀g) of the mass 306. As shown in FIG. 3E, the five bottommost rows of protrusions of the mass 306 may no longer be supported by the pads.

Accordingly, most of the water inside the reservoir may be pushed out step by step at a low pumping rate.

The beauty of such design is that the flow rate may depend on the pumping or evaporation rate of the exposed fluid or water absorbing material 308 only, which can be controlled by design.

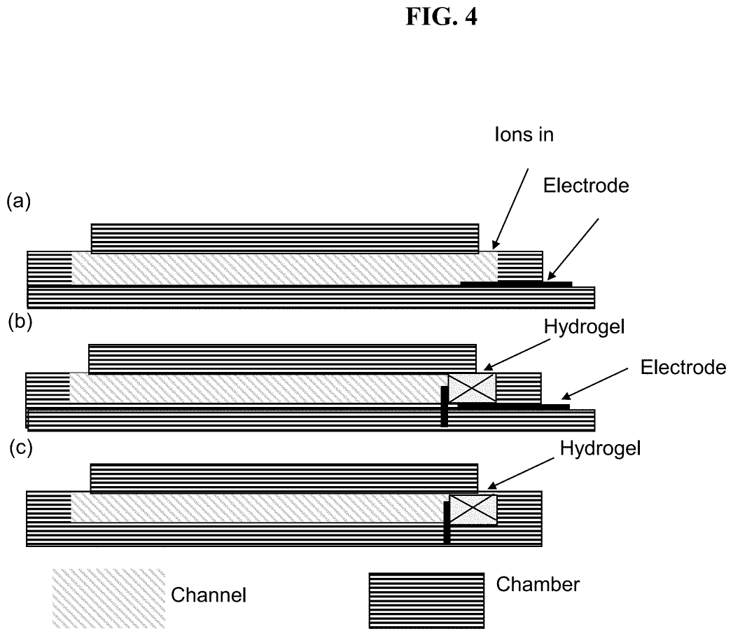

FIG. 4 shows (a) a cross-sectional schematic of an ion sensor chip according to various embodiments (the chip may have a fluidic channel and ions may be dissolved and be carried to the electrodes through an opening) for use in conjunction with the passive fluid transfer system according to various embodiments; (b) a cross-sectional schematic of the ion sensor chip including a hydrogel for locking water and maintaining the humidity of the sensor electrodes according to various embodiments; and (c) a cross-sectional schematic of a test chamber for water retention experiment according to various embodiments.

The sensor electrodes are covered with hydrogel, and a fluidic channel is connected to the hydrogel. The hydrogel is exposed to the environment. Ions can diffuse onto the electrode and water can evaporate.

Conductivity measurement was done to evaluate the water retention effect of the hydrogel using the above setup. A chip with two gold (Au) electrodes was connected with the fluidics chamber.

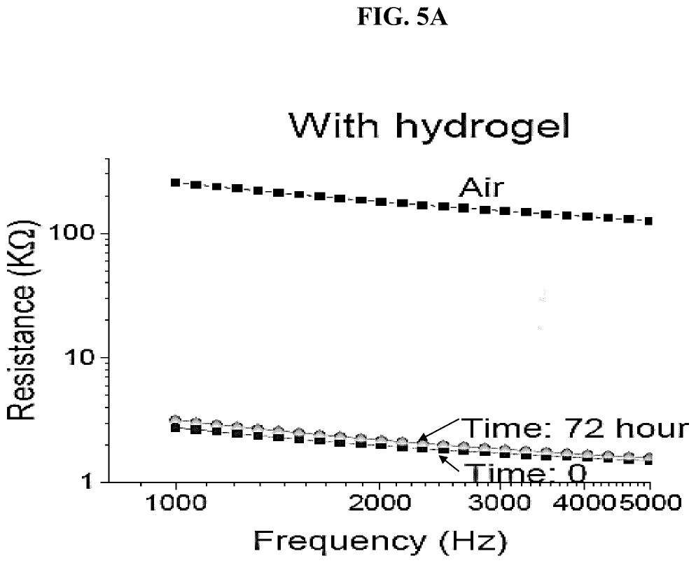

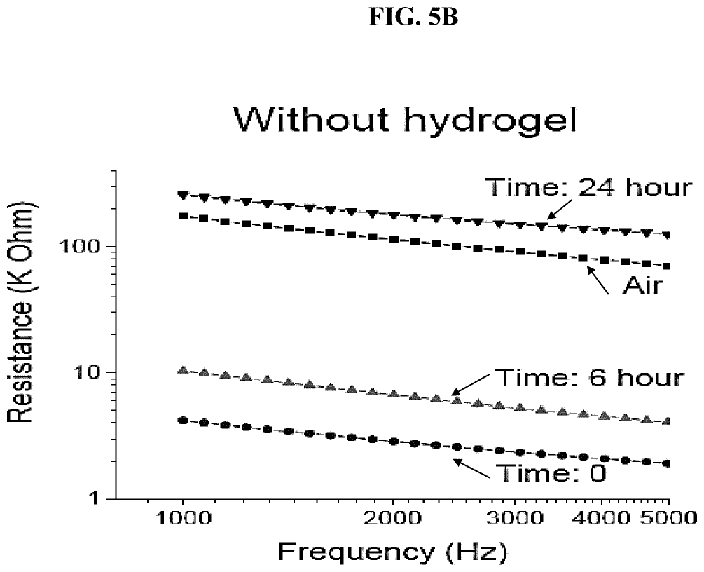

FIG. 5A shows a plot of resistance (in kilo-ohms or kΩ) as a function of frequency (in hertz or Hz) showing the conductivity measurements of the sensing electrodes with hydrogel sealing according to various embodiments. FIG. 5B shows a plot of resistance (in kilo-ohms or kΩ) as a function of frequency (in hertz or Hz) showing the conductivity measurements of the sensing electrodes without hydrogel sealing according to various embodiments.

When no water is inside the chamber, the resistance is high (larger than 100 kΩ at 1 kHz). When a potassium chloride (KCl) solution (10 mM) is introduced into the chamber, the resistance is below 5 kΩ at 1 kHz. For the chip without hydrogel, the resistance increases with buffer evaporation. The resistance increased to above 100 kΩ (1 kHz) after around one day when the solution on top of the electrodes is dried out, indicating loss of the functionality of the device. For the device with hydrogel injected into the chamber above the sensor electrodes, there is no drastic resistance change three days after 500 μl of KCl solution is provided in the fluidic channel. The results clearly demonstrate the moisture retention capability of the hydrogel.

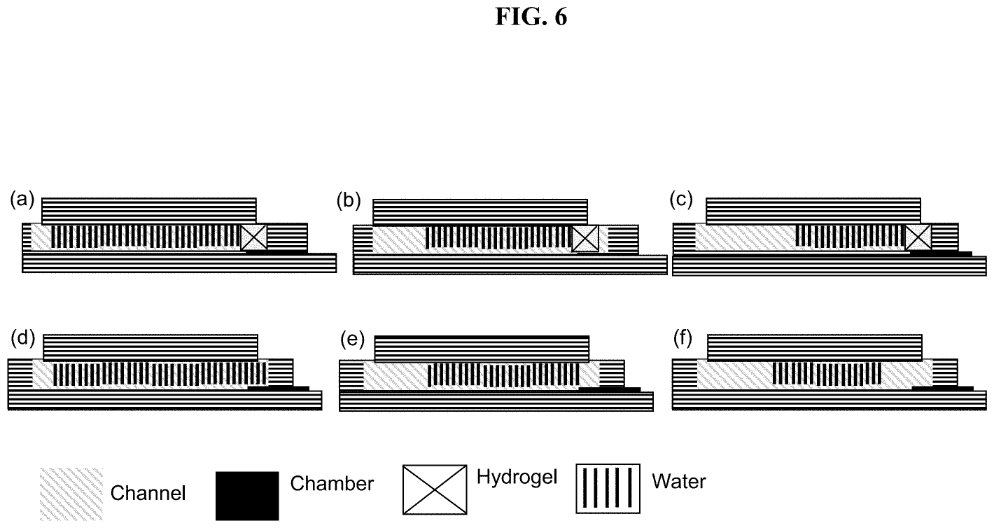

FIG. 6 shows (a) a cross-sectional schematic of the ion sensor chip including hydrogel according to various embodiments at start of experiment (T=0); (b) a cross-sectional schematic of the ion sensor chip shown in (a) according to various embodiments after 40 hours (T=40 h); (c) across-sectional schematic of the ion sensor chip shown in (a) according to various embodiments after 112 hours (T=112 h); (d) a cross-sectional schematic of the ion sensor chip without hydrogel according to various embodiments at start of experiment (T=0); (e) a cross-sectional schematic of the ion sensor chip shown in (d) according to various embodiments after 40 hours (T=40 h); and (f) a cross-sectional schematic of the ion sensor chip shown in (d) according to various embodiments after 112 hours (T=112 h).

When no hydrogel is introduced, the water will evaporate slowly, and the exposed area will be dried out in around one day (d/4, e/5, f/6). This phenomenon may not be affected by the solution volume inside the fluidics channel, which means adding more water in the fluidics channel does not decrease the rate of drying, as drying happens in the exposed area.

When a small amount of hydrogel is put inside the open area of the fluidic chamber (a/1), water may evaporate similarly. However, once evaporation happened, the hydrogel may withdraw more water from the fluidic channel (b/2, c/3). 500 μl of KCl solution may last for more than 5 days at ambient condition. A larger volume of water inside the chamber may last a longer time. Assume the same situation, 40 ml of water may last more than one year.

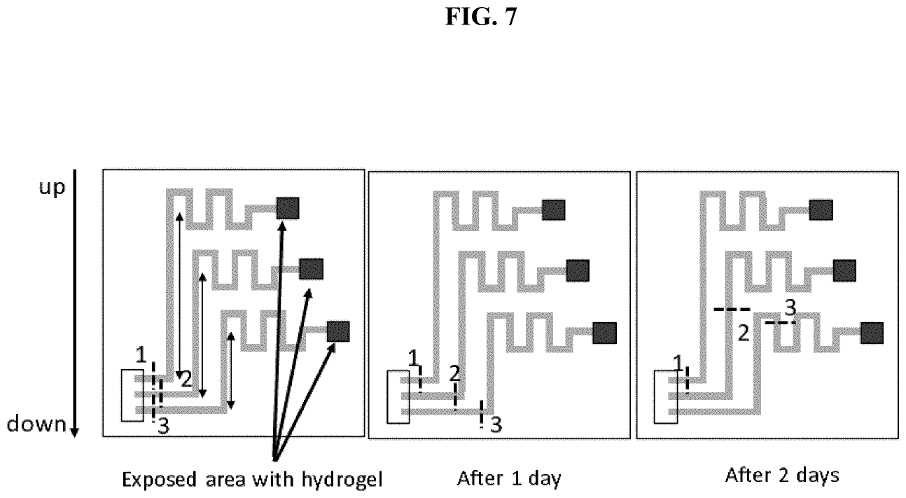

Due to the water-absorbent characteristics of the hydrogel, water can be pumped up at a level lower than the hydrogel. FIG. 7 shows schematics of three fluidic channels according to various embodiments, each with a different initial water level. The water levels are different from the level of the hydrogel. For channel 1, the initial reservoir level is 35 mm below hydrogel. For channel 2, the initial reservoir level is 25 mm below hydrogel. For channel 3, the initial reservoir level is 15 mm below hydrogel. The hydrogel can withdraw water from the reservoir for both channel 2 and 3, but not channel 1. In order to continuously pump the water from the reservoir, there may be a need to restore the water level inside the reservoir. This is realized by the self-adjustable reservoir described before which use a weight and flexible pads to push the water out slowly and at the same time to keep the water level inside the reservoir to be similar to the hydrogel height.

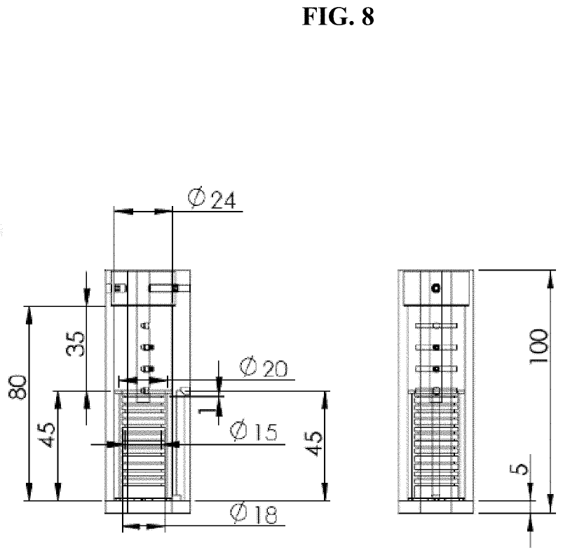

FIG. 8 shows (left) a schematic illustrating a cross-sectional side view of the passive water transfer system according to various embodiments; and (right) a schematic illustrating a cross-sectional front view of the passive water transfer system according to various embodiments. In one implementation, the system may have a cross-section of 34 mm by 31 mm, a height of 100 nm and may be capable of transferred about 9 ml of water. The water transferred may last about 2-3 months.

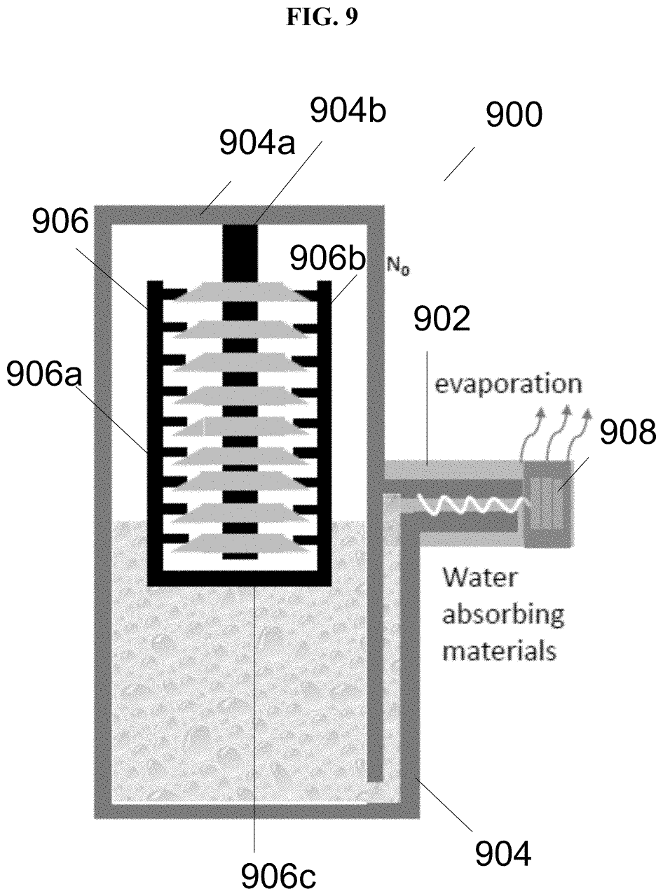

FIG. 9 is a schematic illustrating a fluid transfer system 900 according to various embodiments. The fluid transfer system 900 may include a chamber 902 containing a fluid absorbing material 908 in contact with a device. The fluid transfer system 900 may also include a reservoir 904 including rows of bendable support members, the reservoir 904 connected to the chamber 902 for transport of a fluid from the reservoir 904 to the fluid absorbing material 908. The fluid transfer system 900 may further include a mass 906 including rows of protrusions configured to engage with the rows of bendable support members, the mass 906 configured to be arranged in the reservoir 904 such that forces acting on the mass 906 include a gravitational force on the mass 906, a floating force exerted by the fluid on the mass 906, and a contact force exerted by the rows of bendable support members on the mass 906.

As shown in FIG. 9, the reservoir 904 may include a lid 904a and a column 904b suspended from the lid 904a. The rows of bendable support members may extend from the column 904b. The mass 906 may be an U-shaped structure having two arms 906a, 906b, with the rows of protrusions extending from the two arms 906a, 906b. The first arm 906a may have a first column of protrusions and the second arm 906b may have a second column of protrusions. The first column of protrusions and the second column of protrusions may face each other. The two arms 906a, 906b may be joined by a connector 906c at the bottom.

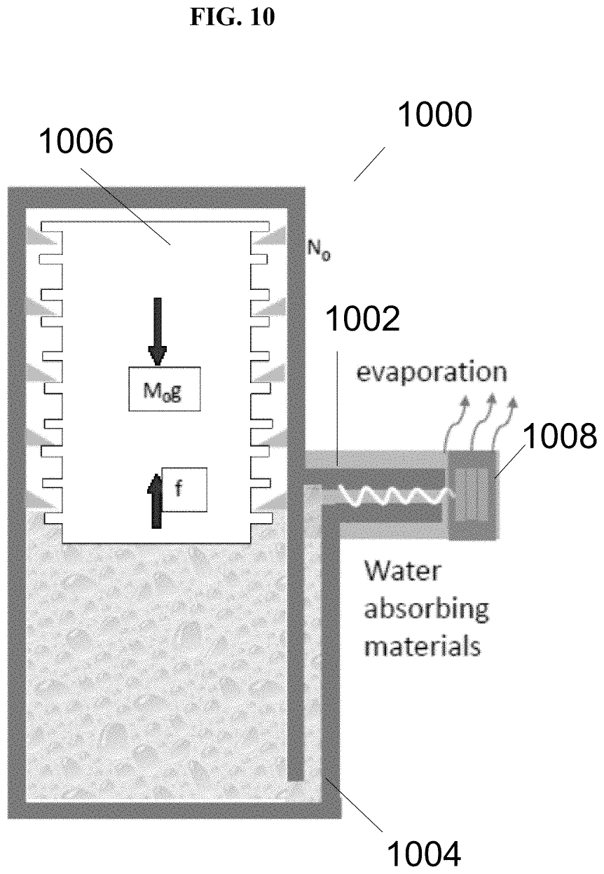

FIG. 10 is a schematic illustrating a fluid transfer system 1000 according to various embodiments. The fluid transfer system 1000 may include a chamber 1002 containing a fluid absorbing material 1008 in contact with a device. The fluid transfer system 1000 may also include a reservoir 1004 including rows of bendable support members, the reservoir 1004 connected to the chamber 1002 for transport of a fluid from the reservoir 1004 to the fluid absorbing material 1008. The fluid transfer system 1000 may further include a mass 1006 including rows of protrusions configured to engage with the rows of bendable support members, the mass 1006 configured to be arranged in the reservoir 1004 such that forces acting on the mass 1006 include a gravitational force on the mass 1006, a floating force exerted by the fluid on the mass 1006, and a contact force exerted by the rows of bendable support members on the mass 1006.

As shown in FIG. 10, the number of rows of bendable support members of the reservoir 1004 may be less than the number of rows of protrusions of the mass 1006.

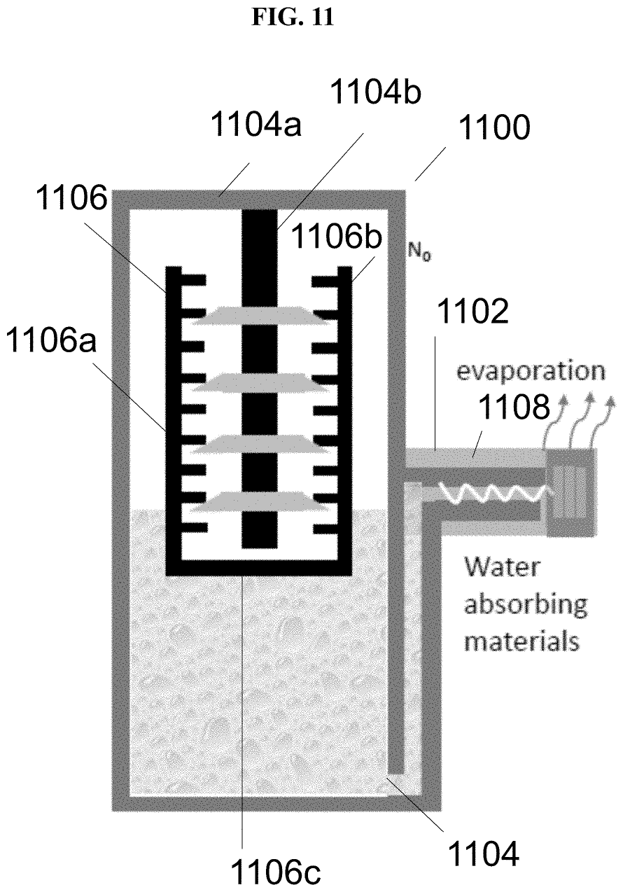

FIG. 11 is a schematic illustrating a fluid transfer system 1100 according to various embodiments. The fluid transfer system 1100 may include a chamber 1102 containing a fluid absorbing material 1108 in contact with a device. The fluid transfer system 1100 may also include a reservoir 1104 including rows of bendable support members, the reservoir 1104 connected to the chamber 1102 for transport of a fluid from the reservoir 1104 to the fluid absorbing material 1108. The fluid transfer system 1100 may further include a mass 1106 including rows of protrusions configured to engage with the rows of bendable support members, the mass 1106 configured to be arranged in the reservoir 1104 such that forces acting on the mass 1106 include a gravitational force on the mass 1106, a floating force exerted by the fluid on the mass 1106, and a contact force exerted by the rows of bendable support members on the mass 1106.

As shown in FIG. 11, the reservoir 1104 may include a lid 1104a and a column 1104b suspended from the lid 1104a. The rows of bendable support members may extend from the column 1104b. The mass 1106 may be an U-shaped structure having two arms 1106a, 1106b, with the rows of protrusions extending from the two arms 1106a, 1106b. The first arm 1106a may have a first column of protrusions and the second arm 1106b may have a second column of protrusions. The first column of protrusions and the second column of protrusions may face each other. The two arms 1106a, 1106b may be joined by a connector 1106c at the bottom.

As shown in FIG. 11, the number of rows of bendable support members of the reservoir 1104 may be less than the number of rows of protrusions of the mass 1106.

By “comprising” it is meant including, but not limited to, whatever follows the word “comprising”. Thus, use of the term “comprising” indicates that the listed elements are required or mandatory, but that other elements are optional and may or may not be present.

By “consisting of” is meant including, and limited to, whatever follows the phrase “consisting of”. Thus, the phrase “consisting of” indicates that the listed elements are required or mandatory, and that no other elements may be present.

The inventions illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

By “about” in relation to a given numerical value, such as for temperature and period of time, it is meant to include numerical values within 10% of the specified value.

The invention has been described broadly and generically herein. Each of the narrower species and sub-generic groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non- limiting examples. In addition, where features or aspects of the invention are described in terms of Markush groups, those skilled in the art will recognize that the invention is also thereby described in terms of any individual member or subgroup of members of the Markush group.

1. A fluid transfer system comprising:
a chamber containing a fluid absorbing material in contact with a device;
a reservoir comprising rows of bendable support members, the reservoir connected to the chamber for transport of a fluid from the reservoir to the fluid absorbing material; and
a mass comprising rows of protrusions configured to engage with the rows of bendable support members, the mass configured to be arranged in the reservoir such that forces acting on the mass comprise a gravitational force on the mass, a floating force exerted by the fluid on the mass, and a contact force exerted by the rows of bendable support members on the mass.

2. The fluid transfer system according to claim 1,
wherein the gravitational force acting on the mass is initially balanced by a sum of the floating force exerted by the fluid on the mass and the contact force exerted by the rows of bendable support members on the mass;
wherein the rows of bendable support members are configured such that a first row of bendable support members of the rows of bendable support members initially supporting a row of protrusions of the rows of protrusions bends as the floating force exerted by the fluid decreases, causing the gravitational force acting on the mass to be more than the sum of the floating force exerted by the fluid on the mass and the contact force exerted by the rows of bendable support members on the mass; and
wherein bending of the first row of bendable support members allows the row of protrusions to move past the first row of bendable support members so that the row of protrusions is supported by a second row of bendable support members of the rows of bendable support members below the first row of bendable support members, thereby allowing the mass to move down relative to the reservoir.

3. The fluid transfer system according to claim 2, wherein the floating force exerted by the fluid on the mass decreases as the fluid travels from the reservoir to the fluid absorbing material to replace the fluid lost via evaporation.

4. The fluid transfer system according to claim 2, wherein the floating force exerted by the fluid on the mass increases and the contact force exerted by the rows of bendable support members on the mass decreases as the mass moves down relative to the reservoir.

5. The fluid transfer system according to claim 1,
wherein the fluid is water; and
wherein the fluid absorbing material is a hydrophilic material.

6. The fluid transfer system according to claim 5, wherein the hydrophilic material is a hydrogel.

7. The fluid transfer system according to claim 1, further comprising:
the device.

8. The fluid transfer system according to claim 1, wherein the device is an ion sensor electrode.

9. The fluid transfer system according to claim 1,
wherein the reservoir comprises one or more walls; and
wherein the rows of bendable support members extend from the one or more walls.

10. The fluid transfer system according to claim 1,
wherein the reservoir comprises:
a lid; and
a column suspended from the lid; and
wherein the rows of bendable support members extend from the column.

11. A method of forming a fluid transfer system, the method comprising:
providing a chamber containing a fluid absorbing material in contact with a device;
connecting a reservoir to the chamber for transport of a fluid from the reservoir to the fluid absorbing material, the reservoir comprising rows of bendable support members; and
providing a mass comprising rows of protrusions configured to engage with the rows of bendable support members, the mass configured to be arranged in the reservoir such that forces acting on the mass comprise a gravitational force on the mass, a floating force exerted by the fluid on the mass, and a contact force exerted by the rows of bendable support members on the mass.

12. The method according to claim 11,
wherein the gravitational force acting on the mass is initially balanced by a sum of the floating force exerted by the fluid on the mass and the contact force exerted by the rows of bendable support members on the mass;
wherein the rows of bendable support members are configured such that a first row of bendable support members of the rows of bendable support members initially supporting a row of protrusions of the rows of protrusions bends as the floating force exerted by the fluid decreases, causing the gravitational force acting on the mass to be more than the sum of the floating force exerted by the fluid on the mass and the contact force exerted by the rows of bendable support members on the mass; and
wherein bending of the first row of bendable support members allows the row of protrusions to move past the first row of bendable support members so that the row of protrusions is supported by a second row of bendable support members of the rows of bendable support members below the first row of bendable support members, thereby allowing the mass to move down relative to the reservoir.

13. The method according to claim 12, wherein the floating force exerted by the fluid on the mass decreases as the fluid travels from the reservoir to the fluid absorbing material to replace the fluid lost via evaporation.

14. The method according to claim 12, wherein the floating force exerted by the fluid on the mass increases and the contact force exerted by the rows of bendable support members on the mass decreases as the mass moves down relative to the reservoir.

15. The method according to claim 11,
wherein the fluid is water; and
wherein the fluid absorbing material is a hydrophilic material.

16. The method according to claim 15, wherein the hydrophilic material is a hydrogel.

17. The method according to claim 11, further comprising:
providing the device.

18. The method according to claim 11, wherein the device is an ion sensor electrode.

19. The method according to claim 11,
wherein the reservoir comprises one or more walls; and
wherein the rows of bendable support members extend from the one or more walls.

20. The method according to claim 11,
wherein the reservoir comprises:
a lid; and
a column suspended from the lid; and
wherein the rows of bendable support members extend from the column.
